(19) United States
(12) Patent Application Publication
(10) Pub. No.: US 2022/0290105 A1
(43) Pub. Date: Sep. 15, 2022

(54) MESENCHYMAL STEM CELLS WITH ENHANCED THERAPEUTIC PROPERTIES

(71) Applicants: CELLS FOR CELLS S.A., Santiago (CL); CONSORCIO REGENERO, S.A., Santiago (CL); UNIVERSIDAD DE LOS ANDES, Santiago (CL); UNIVERSITÉ DE MONTPELLIER, Montpellier (FR)

(72) Inventors: Patricia LUZ CRAWFORD, (US); Rafael CONTRERAS LÓPEZ, (US); Roberto ELIZONDO VEGA, (US); Farida DJOUAD, (US)

(21) Appl. No.: 17/756,456

(22) PCT Filed: Nov. 26, 2020
(86) PCT No.: PCT/IB2020/061190

§ 371 Date: May 25, 2022

(30) Foreign Application Priority Data
Nov. 26, 2019 (EP) 19211428.8

(51) Int. Cl. C12N 5/0775; A61K 35/28; A61P 19/08; A61K 35/51; A61K 38/18; A61K 9/00

(52) U.S. Cl. CPC C12N 5/0665; A61K 35/28; A61P 19/08; A61K 35/51; A61K 38/1825; A61K 9/0019

(57) ABSTRACT

Mesenchymal stem cells with enhanced therapeutic properties. The present invention refers to a method for obtaining mesenchymal stem cells (MSCs) with enhanced immunosuppression, chondroprotection and chondrodifferentiation properties, and enhanced capability to promote the proliferation of chondrocytes. The method comprises contacting the MSCs; culturing MSC in a induction media supplemented with 0.01 to 100 μg/ml of an ATP-synthase inhibitor; and removing the induction media to obtain MSC with enhanced activity. Moreover, the present invention also refers to the MSCs themselves and to their use for cell therapy purposes, preferably for treating autoimmune and/or osteoarticular diseases.

### FIELD OF THE INVENTION

The present invention pertains to the medical field. Particularly, the present invention can be encompassed within the field of biomedicine and tissue engineering. More specifically, the present invention refers to a method for obtaining mesenchymal stem cells (MSCs) with enhanced immunosuppression, chondroprotection and chondrodifferentiation properties, and enhanced capability to promote the proliferation of chondrocytes. Moreover, the present invention also refers to the MSCs themselves and to their use for cell therapy purposes, preferably for treating autoimmune and/or osteoarticular diseases.

### STATE OF THE ART

MSCs form a heterogeneous population, capable of retaining their multipotent capacity and releasing various factors and cytokines such as vascular endothelial growth factor (VEGF), fibroblast growth factor β (FGF-β), insulin growth factor (IGF) and hepatocyte growth factor (HGF), for promoting angiogenesis, inhibit apoptosis and induce cell proliferation. These capabilities have proposed that MSCs have the physiological role of maintaining tissue homeostasis by secreting factors in a paracrine fashion and producing cell proliferation and replacement, allowing cell survival during aging, tissue damage or disease. These properties have defined MSCs as a powerful therapeutic tool in the field of regenerative medicine. In addition, it has been shown that MSCs have a strong immunosuppressive effect, which has proposed their use as cell therapy for various autoimmune diseases such as rheumatoid arthritis (RA), autoimmune encephalitis, systemic lupus erythematosus, autoimmune myocarditis or graft-versus-host disease (GVHD) among others. However, phase III clinical trials using MSCs to treat inflammatory/autoimmune disorders have not always shown therapeutic effects. The absence of beneficial effects after the administration of MSCs in clinical trials may be due to the lack of an appropriate stimulus in the microenvironment of the MSCs. Therefore, there is a need to enhance the therapeutic effect of the MSCs for an adequate therapeutic use. In the last years, different strategies have been suggested to induce certain key characteristics that increase or promote their therapeutic potential. For example, the pre-treatment of MSCs with vitamin E has been proposed to increase resistance to reactive oxygen species (ROS). On the other hand, the use of proinflammatory cytokines has also been proposed to promote the immunosuppressive effect of MSCs. Finally, the induction of hypoxic conditions as well as the 3D culture of MSCs has shown an improvement in their proliferation and differentiation potential towards cartilage.

However, the above-motioned proposals are not able to induce regeneration of chondrocytes in the damage joint.

Thus, there is still an unmet medical need of finding new and effective strategies or protocols able to successfully promote the therapeutic potential of the MSCs. In fact, nowadays, there are no therapies approved by the FDA based on allogenic MSCs for the treatment of diseases such as osteoarthritis, rheumatoid arthritis, nor are there pending regulatory approvals from other agencies (EMA, FDA, and others) to market treatments with MSCs for such use.

The present invention is then focused on solving the above-cited problem and it is herein provided a method for obtaining MSCs with improved therapeutic properties that can be successfully used for treating autoimmune and/or osteoarticular diseases, wherein the method comprises pre-treating the MSCs with ATP-Synthase inhibitors.

In the state of art, we found that some ATP-Synthase inhibitors have been used together with MSCs, but in a different way and with a different purpose than the method and purpose of the present invention. For example, in a previous application by the inventors, WO2019/100175, the use of Pterostilbene as an oxidative protector in the pre-treatment embodiments was described, especially before exposure the MSCs to oxidative stress. Wherein, in our previous application the method described didn't achieve MSCs with enhanced immunosuppression, chondroprotection and chondrodifferentiation properties, neither enhanced capability to promote the proliferation of chondrocytes, as we do in the present invention. Moreover, the invention protected here include the step of removing the induction media to obtain MSC with enhanced activity, step not included in the WO2019/100175.

Buhrmann et al (Journal of Biological Chemistry Volume 289, Number 32, p 22048-62, Aug. 8, 2014) describes an in vitro treatment for MSC using a composition containing resveratrol (5 μM), IL-1β (10 ng/ml), NAM (10 mM) or BMS-345541 (5 μM) for 14 days or a pretreatment with resveratrol (5 μM) for 4 hours followed by a co-treatment with IL-1β (10 ng/ml), NAM (10 mM) or BMS-345541 (5 μM) or transfected with 0.5 μM SIRT1-SO or SIRT1-ASO in a medium of chondrogenic induction for 14 days. This document acknowledges that it is completely different from the method of the invention, wherein only an ATP synthase inhibitor is used, which may or may not be resveratrol, for 6 to 24 hours without the need for any additional factor and without the need of cultivating the MSCs in a chondrodifferentiation-inducing medium (see FIG. 5).

Therefore, the documents in the state of the art do not anticipate the invention.

### DESCRIPTION OF THE INVENTION

### Brief Description of the Invention

The present invention refers to a method for obtaining MSCs with enhanced immunosuppression, chondroprotection and chondrodifferentiation properties, and enhanced capability to promote the proliferation of chondrocytes. Moreover, the present invention also refers to the MSCs themselves and to their use for cell therapy purposes, preferably for treating autoimmune and/or osteoarticular diseases.

During recent years, the inventors of the present invention have been looking for a protocol that promotes the therapeutic potential of the MSCs by enhancing their resistance to free radicals, in order to have a greater number of functional cells in the area of damage and inflammation, increasing their immunosuppressive capacity to manage the inflammation of the area to be treated and finally to have a greater chondroprotective and regenerative effect of hyaline cartilage.

Finally, the inventors have surprisingly found that when the glycolytic metabolic status of the MSCs is increased by treating the MSCs with ATP-Synthase inhibitors, MSCs with improved therapeutic properties are obtained. Specifically, the MSCs obtained once they are submitted to a treatment with an ATP-Synthase inhibitor (hereinafter MSCs of the invention) show enhanced immunosuppression, chondroprotection and chondrodifferentiation properties, and enhanced capability to promote the proliferation of chondrocytes.

In fact, such as it is demonstrated in the present invention (see Example 2), the MSCs of the invention can be used, once they are treated with an ATP-Synthase inhibitor, for treating autoimmune and/or osteoarticular diseases. The treatment with the MSCs of the invention will not only significantly reduce pain, but also stop chondrogenic damage, promote the proliferation of chondrocytes, reduce the inflammation and stimulate the regeneration of damaged cartilage. Particularly, such as depicted in FIG. 1, MSCs treated with oligomycin show an improved effect over MSCs treated with atovaquone or potassium cyanide (KCN). Specifically, MSCs treated with oligomycin show an increase in their suppressive potential as compared with atovaquone or KCN.

Thus, the first embodiment of the present invention refers to MSCs treated with an ATP-synthase inhibitor; for use in the treatment of osteoarticular and/or autoimmune diseases. In other words, this first embodiment of the invention refers to MSCs for use in the treatment of osteoarticular and/or autoimmune diseases, characterized in that the MSCs are treated with an ATP-synthase inhibitor. Alternatively, this first embodiment of the invention refers to a method for treating osteoarticular and/or autoimmune diseases which comprises the administration of a therapeutically effective amount of the MSCs of the invention (i.e. MSCs treated with an ATP-synthase inhibitor). In a preferred embodiment, the ATP-synthase inhibitor is administered to the MSCs before the MSCs are administered to the patient (thus MSCs pre-treated with the ATP-synthase inhibitor are administered to the patient).

In a preferred embodiment, the MSCs of the invention can regenerate damaged cartilage or to generate new cartilage. So, this preferred embodiment refers to the MSCs of the invention for use in the regeneration of damaged cartage or in the generation of new cartilage. Alternatively, this embodiment of the invention refers to a method for the regeneration of damaged cartilage or for the generation of new cartilage, which comprises the administration of a therapeutically effective amount of the MSCs of the invention.

In a preferred embodiment, the MSCs of the invention can be used to treat skin injuries. For example, the MSCs of the invention can be used to improve or promote the wound healing process, thus repairing the skin, and tissues located under the skin, after injury. Moreover, the MSCs of the invention can be used for treating skin ulcers such as pressure ulcers, venous or arterial skin ulcers or neuropathic skin ulcers (diabetic foot ulcers). Thus, this preferred embodiment refers to the MSCs of the invention for use in the treatment of said skin injuries. Alternatively, this embodiment of the invention refers to a method for treating said skin injuries which comprises the administration of a therapeutically effective amount of the MSCs of the invention.

The second embodiment of the present invention refers to the in vitro use of an ATP-synthase inhibitor for obtaining MSCs with enhanced immunosuppression, chondroprotection and chondrodifferentiation properties, and enhanced capability to promote the proliferation of chondrocytes.

The third embodiment of the present invention refers to an in vitro method for obtaining MSCs with enhanced immunosuppression, chondroprotection and chondrodifferentiation properties, and with enhanced capability to promote the proliferation of chondrocytes, which comprises contacting MSCs with an ATP-synthase inhibitor.

The fourth embodiment of the present invention refers to the MSCs of the invention obtained by means of the in vitro method described above. In a preferred embodiment, the MSCs of the invention are characterized by having ATP-synthase inhibitors inside the cells, particularly inside the mitochondria.

The fifth embodiment of the present invention refers to a pharmaceutical composition comprising MSCs obtained by means of the in vitro method described above, which are preferably characterized by having ATP-synthase inhibitors inside the cells, particularly inside the mitochondria and optionally pharmaceutically acceptable carriers. In other words, this embodiment refers to a pharmaceutical composition comprising MSCs treated with an ATP-synthase inhibitor and, optionally, pharmaceutically acceptable carriers.

In a preferred embodiment, the MSCs of the invention can be used for treating osteoarticular diseases selected from the group comprising (non-exhaustive list): osteoarthritis, musculoskeletal disorders, rheumatoid arthritis or osteoporosis; preferably osteoarthritis or rheumatoid arthritis.

On the other hand, the MSCs of the invention can be used for treating autoimmune diseases selected from the group comprising (non-exhaustive list): rheumatoid arthritis, systemic lupus erythematosus, inflammatory bowel disease, multiple sclerosis, type 1 diabetes mellitus, guillain-barre syndrome, chronic inflammatory demyelinating polyneuropathy, psoriasis, Graves' disease, Hashimoto's thyroiditis, myasthenia gravis, vasculitis, Sjögren's syndrome, Hashimoto's thyroiditis, pernicious anemia, celiac disease or graft versus host disease.

In a preferred embodiment, the ATP-synthase inhibitor enhances the transmigration and migration potential of stem cells.

In a preferred embodiment, the MSCs of the invention can be administered following any route of administration known in the prior art for MSCs. The preferred routes of administration are: intravenous, intramuscular, subcutaneous and/or transdermal administration routes, preferably intravenous routes.

In a preferred embodiment, the ATP-synthase inhibitor enhances the immunosuppression carried out by the mesenchymal stem cells, enhances the chondroprotection of mesenchymal stem cells, promotes the chondrodifferentiation of mesenchymal stem cells and/or enhances the mesenchymal stem cells to promote the proliferation of chondrocytes.

In a preferred embodiment, the MSCs of the invention are derived from (non-exhaustive list): umbilical cord (UC-MSCs), adipose tissue, menstrual fluid, bone marrow, dental pulp, blood, endometrial tissue, peripheral blood, placental tissue or they are induced pluripotent stem cells.

In a preferred embodiment, the ATP-synthase inhibitor used for treating the MSCs is selected from the list comprising: Tentoxin, Efrapeptins, Substrate analogs (DCCD, CMCD, EEDQ,NBD-Cl (βE), Azide), Aurovertins, Asteltoxin, Resveratrol, Piceatannol, α-Helical basic peptides, Bz-423, Estrogens, Angiostatin, Enterostatin, Citreoviridin, Quinacrine mustard, Bathophenanthroline-metal chelates, Oligomycin, Venturicidin, DCCD, NCCD, R207910, Organotin compounds or Ossamycin; preferably: Oligomycin, Venturicidin, Resveratrol, Piceatannol, Tributyltin or Pterostilbene; and more preferably Oligomycin.

Nevertheless, such as it is shown in the present invention (see for example FIG. 2 and FIG. 3), it is important to note that members pertaining to different families of inhibitors of the ATP-synthase complex have been assayed in the present invention, and all of them have given rise to successful results. This means that the key point of the invention is that the therapeutic properties of the MSCs are improved once the ATP-Synthase is inhibited, irrespective of the ATP-Synthase inhibitor which is finally used. In other words, any ATP-synthase inhibitor could be successfully used in the present invention for achieving MSCs with improved therapeutic properties. In order to give specific examples of ATP-Synthase inhibitors which could be used according to the present invention, the document [Sangjin Hong et al. 2008. ATP Synthase and the Actions of Inhibitors Utilized To Study Its Roles in Human Health, Disease, and Other Scientific Areas. Microbiol Mol Biol Rev. 2008 December; 72(4): 590-641. doi: 10.1128/MMBR.00016-08. PMCID: PMC2593570. PMID: 19052322] is herein incorporated in its entirety to the description of the present invention, making special reference to the FIG. 11 of the document wherein the preferred ATP-synthase inhibitor is illustrated.

Please note that there are different kinds of ATP-Synthase inhibitors, due to the fact that the ATP-Synthase is a complex molecule that has several inhibitor binding sites. The main ATP-synthase inhibitors as drug scaffolds are Tentoxin, Efrapeptins, Substrate analogs (DCCD, CMCD, EEDQ,NBD-Cl (βE), Azide), Aurovertins, Asteltoxin, Resveratrol, Piceatannol, α-Helical basic peptides, Bz-423, Estrogens, Angiostatin, Enterostatin, Citreoviridin, Quinacrine mustard, Bathophenanthroline-metal chelates, Oligomycin, Venturicidin, DCCD, NCCD, R207910, Organotin compounds, and Ossamycin.

More specifically, the following ATP-Synthase inhibitors are identified, and any of them could be used in the present invention in order to obtain MSCs with improved therapeutic properties:
Peptide Inhibitors such as alpha-helical basic peptide inhibitors, angiostatin and enterostatin, tentoxin and tentoxin analogs, leucinostatins, efrapeptins.
Polyphenolic Phytochemicals, Estrogens, and structurally related compounds such as Stilbenes, specifically but not limited to Resveratrol (3,4,5-Stilbenetriol; 3,4,5-trihydroxystilbene), Piceatannol (3,5,3,4-Tetrahydroxystilbene;3-hydroxyresveratol), Diethylstilbestrol (DES, Diethylstilbestrol; (E)-4,4-(1,2-diethyl-1,2-ethenediyl)bisphenol; 4,4-dihydroxydiethylstilbene; (E)-3,4-bis(4-hydroxyphenyl)-3-ascic; Acnestrol; Antigestil; Comestrol; Cyren; Desma; Dibestrol; Distilbene; Estrobene; Pabestrol; Stilbetin; Vagestrol) 4-Acetamido-4-isothiocyanostilbene 2,2-disulfonate (SITS), 4,4-Di-isothiocyanatostilbene-2,2-disulfonic acid; diisothiocyanatostilbene-2,2-disulfonic acid.
Other compounds such as Flavones, such as Quercetin, specifically including but not limited to 3,3,4,5,7-Pentahydroxyflavone; natural yellow 10; meletin; flavin;meletin; quercetol; Xanthaurine. Kaempferol, specifically but not limited to Kempferol; campherol; indigo yellow; nimbecetin; pelargidenolon; populnetin; rhamnolutein; 3,4,5,7-tetrahydroxyflavone; trifolitin. Morin, specifically but not limited to, 2,3,4,5,7-Pentahydroxyflavone; 2,4,5,7-tetrahydroxyflavan-3-ol; 3,5,7,2,4-pentahydroxyflavonol; al-morin; aurantica; calico yellow; osage orange. Apigenin, specifically but not limited to 4,5,7-Trihydroxyflavaone; 2-(p-hydroxyphenyl)-5,7-dihydroxychromone; apigenol; chamomile; spigenin.
Other compounds such as Isoflavones such as Genistein, specifically including but not limited to 4,5,7-Trihydroxyisoflavone; genisteol; genisterin; prunetol; sophoricol; differenol A. Biochanin specifically but not limited to A Biochanin; 4-methylgenistein; 5,7-dihydroxy-4-methoxyisoflavone; CCRIS 5449; 5,7-dihydroxy-4-methoxyisoflavone. Daidzein, specifically but not limited to 4,7-Dihydroxyisoflavone; daidzeol; 7-hydroxy-3-(4-hydroxyphenyl)-4-benzopyrone.
Other compounds such as Polyphenolic Phytochemicals, comprising ECG, ECGC, GSPE, Curcumin, Phloretin, Theaflavin, Tannic acid.
Other compounds such as Steroidal Estradiols and Estrogen Metabolites, comprising 4-hydroxyestradioland 2-hydroxyestradiole, 17-α-Estradiol, 17-β-Estradiol, α-Zearalenol, β-Zearalanol.
Organotin Compounds and Structural Relatives such as Organotin compounds that contain R4Sn, R3SnX, R2SnX2, and RSnX3, Tributyltin chloride, Tricyclohexyltin hydroxide, Triethyltin sulfate, Triphenyltin chloride, Dimethyltin 3-hydroxyflavone chloride, Diethyltin 3-hydroxyflavone chloride, Dibutyltin 3-hydroxyflavone bromide, Dioctyltin 3-hydroxyflavone chloride, Diphenyltin 3-hydroxyflavone chloride, Diethyltin 3,5,7,2,4-pentahydroxy flavone chloride, Dibutyltin 3,5,7,2,4-pentahydroxy flavone bromide, Diphenyltin 3,5,7,2,4-pentahydroxy flavone chloride, Tributyltin 3-hydroxyflavone, Triethyllead.
Other compounds such as Polyenic α-Pyrone including, but not limited, to Aurovertin (A, B, C, D, E), Citreoviridin, Asteltoxin.
Cationic Inhibitors such as Amphiphilic Cationic Dyes, including, but not limited to, Rhodamine B, rheonine B; rhodamine O; rhodamine S, Rhodamine 123, Rhodamine 6G, Basic rhodamine yellow; rhodamine J, Rosaniline, Magenta Base, Malachite green, Aniline green, benzal green, Victoria green, Brilliant green, Basic green 1, Quinacrine, mepacrine, Quinacrine mustard, Quinacrine mustard dihydrochloride, Acridine orange, rhoduline orange, Coriphosphine, Coriphosphine O, Pyronin Y, Pyronine; Pyronin G, Dequalinium, Safranin O, basic red, safranine T, Nile blue A, Nile blue, Nile blue AX, EtBr, Ethidium bromide; homidium bromide.
Cationic inhibitors Tertiary amine local anesthetics (TALAs) and related compounds, including, but not limited to, Tetracaine, Dicaine, Dibucaine, Cincainum, Cinchocaine, Dermacaine, dibucainum, Nupercaine, Percamine, Sovcaine, Procaine, Procain, Spinocaine, Lidocaine, Cappicaine, Duncaine, Esracaine, Isicaine, Lidocaine, Maricaine, Xycaine, Xylocaine, Chlorpromazine, phenothiazine, Aminazin, Aminazine, Chlor-Promanyl, Chlorderazin, Chlorpromados, Contomin, Elmarin, Esmind, Fenactil, Largactil, Megaphen, Novomazina, Proma, Phenactyl, Promactil, Propaphenin, Prozil, Psychozine, Sanopron, Thorazine, Torazina, Wintermin, Trifluoperazine, Trifluoromethylperazine, Procainamide, Propranolol.
Other cationic Inhibitors such as organic cations, including, but not limited to, Octyl guanidine, 1-Dansyl amido-3-dimethypropylamine compounds, Cetyltrimethylammonium, Cetrimonium, cetrimonum, cetyltrimethylammonium hexadecyltrimethylammonium, trimethylhexadecylammonium, Spermine, Spermidine, Bathophenan throline-metal (Ru2+, Ni2+, Fe2+) chelate, DPBP-ferrous chelate, PDT-ferrous chelate, Atrazine, Atrazine amino derivative.
Substrates and Substrate analogs such as Phosphate Analogs, including, but not limited to, Arsenate, Aluminum fluoride, Beryllium fluoride, Scandium fluoride, Vanadate (VO₄ 3- and VO₃1-), Orthovanadate, Magnesium fluoride, Sulfite, Thiophosphate, Azide, ANPP.
Other Substrates and Substrate analogs such as Divalent Metal Ions, including, but not limited to, Free Mg2+, Mn2+, Ca2+.
Other Substrates and Substrate analogs such as Purine Nucleotides and Nucleotide Analogs, including, but not limited to, ATP, ADP, GTP, FTP, TNP-ATP, TNP-ADP, TNP-Ado, Lin-benzo-ADP, AP4A, AP5A, AP6A, AMP-PNP, GMP-PNP, IMP-PNP, AMP(CH2)P, RhATP, CrATP or Cr(NH3)4ATP, Co(NH3)4ATP, 3′-O-Acetyl-ATP, 3′-O-Acetyl-ADP, 3′-O-Caproyl-ADP, 3′-O-Enanthyl-ADP, 3′-O-Caprylyl-ADP, F-ADP/DMAN-ADP, F-ATP, 3′-O-(1-Naphthoyl)-ADP/N-ADP, 3′-O-(1-Naphthoyl)-ATP, 3′-O-(2-Naphthoyl)-ADP, B zATP, BzADP, t-Butylacetyl-ADP, 3′-O-Phenylacetyl-ADP, 3′-O-Phenylbutyryl-ADP, 3′-O-Benzoyl-ADP, 3′-O—N-(2-Nitrophenyl)-γ-aminobutyryl-ADP, 3′-O—N-(4-Nitrophenyl)-γ-aminobutyryl-ADP, 3′-O-(1-Naphthylacetyl)-ADP, 3′-O-(2-Naphthyl acetyl)-ADP, 3′-O-(1-Anthranoyl)-ADP, 3′-O-(9-Anthranoyl)-ADP, FSBI, FSBA, FSBξA, AP2-PL, AP3-PL, AP4-PL, oATP, oADP, oAMP, Cibacron blue, BzAF.
Other Substrates and Substrate analogs such as Azidonucleotides, including, but no limited to, 8-Azido-ATP, 8-Azido-ADP, 2-Azido-ATP, 2-Azido-ADP, 2-Azido-TNP-ATP, ANA-ADP, 8-Azido-FSBA, 2-Azido-AMP-PNP, 8-Azido-AMP-PNP, NAP4-ADP, NAP4-AMP-PNP, NAP3-ATP, NAP3-ADP, NAB-ATP, NAB-GTP, ANP-ADP.
Amino acid modifiers such as Amino Group Modifiers, including, but not limited to, Phenylglyoxal, Butanedione, Phenylglyoxal and butanedione, 1-Fluoro-2,4-dinitrobenzene, Dansyl chloride. Other compounds such as Carboxyl Group Modifiers including, but no limited to DCCD, NCCD, CMCD, EDC, EEDQ, Woodward's reagent K. Other Compounds such as Cys and Tyr Residue Modifiers, including, but not limited to, NBD-Cl, Tetranitromethane, Tetan, DFDNB, NEM, Bismuth subcitrate, Omeprazole, Audazol, Omepral, DTNB, PCMB, PCMS, Mersalyl, 2,2′-Dithiobispyridine, N-(7-Dimethylamino-4-methyl-coumarinyl)-maleimide. Other compounds such as His Residue Modifiers including, but not limited to, Diethyl pyrocarbonate, Rose bengal, Iodine.
Physical inhibitory factors such as High Hydrostatic Pressure, UV irradiation, Low temperature.
Miscellaneous inhibitors such as Dicarbopolyborate, Almitrine, 5-Hydroxy-1,2-naphthalene dicarboxylic anhydride, R207910, Spegazzinine, n-Butanol, TCS, Dihydrostreptomycin, Suramin, Bz-423, DMSO, Hypochlorous acid, DDT, Diazoxide, HNB, N-Sulfonyl or N-alkylsubstituted tetrahydrobenzodiazepine derivatives. 4-(N-Arylimidazole)-substituted benzopyran derivatives, N-_1-Aryl-2-(1-imidazolo)ethyl-cyanoguanidine derivatives, N-_1-Aryl-2-(1-imidazolo)ethyl-acylguanidine derivatives, O-[1-Aryl-2-(1-imidazolo)ethyl-thiourethane derivatives, Dio-9 complex, Ethanol, Zinc.

Moreover, the present invention also refers to the use of combinations of at least two ATP-synthase inhibitors in order to obtain MSCs with improved therapeutic properties. According to the present invention, the use of combinations of ATP-synthase inhibitors can give rise to a synergetic effect. This means that an improved result is obtained when a combination of at least two ATP-synthase inhibitors is used, as compared with the use of single ATP-synthase inhibitors. By way of example, FIG. 3 shows the results obtained with different ATP-synthase inhibitors and also with the combination of two of them: oligomycin and resveratrol.

Last but not least, it is important to highlight that a spontaneous differentiation of MSCs into chondrocytes was achieved in the present invention without the need of a differentiation media and just by using a single ATP-synthase inhibitor like, for example, oligomycin. In this way, the present invention offers an alternative, more effective and cheaper approach for the differentiation of MSCs into chondrocytes, which only requires contacting the MSCs with a single ATP-synthase inhibitor. Consequently, the spontaneous differentiation of MSCs into chondrocytes achieved in the present invention, just by contacting MSCs with an ATP-synthase inhibitor, and without the need of a differentiation media, gives rise to an improved method for the treatment of patients since the administration of differentiation media to the patient is neither recommended nor convenient.

In fact, such as it is shown in FIG. 5A, the three-dimensional tissue generated by contacting MSCs with oligomycin in basal media was even greater than the three-dimensional tissue obtained by using the differentiation media alone. Moreover, the treatment using oligomycin in combination with the differentiation media offers similar results as compared with the use of oligomycin alone. Thus, in principle, an improved result is not observed when the differentiation media is incorporated.

FIG. 5B shows that, in basal media, an increased relative expression of the proteoglycan Aggrecan was obtained in MSCs treated only with oligomycin. FIG. 5C shows that a similar relative expression of the proteoglycan Aggrecan was obtained when oligomycin was incorporated in the differentiation media in comparison with the use of the differentiation media alone.

According to FIG. 5D it is important to highlight that a spontaneous change in the production ratio of Collagen II vs Collagen I is observed when an Inhibitor of the ATP Synthase is used with MSC in a basal media. This allows the MSCs to behave just as if they were in a differentiation media, proving that the inventors have developed a method that uses these inhibitors to promote the MSC to differentiate and behave as a chondrocyte.

For the purpose of the present invention the following terms are defined:
The term “comprising” means including, but not limited to, whatever follows the word “comprising”. Thus, use of the term “comprising” indicates that the listed elements are required or mandatory, but that other elements are optional and may or may not be present.
“Consisting of” means including, and limited to, whatever follows the phrase “consisting of”. Thus, the phrase “consisting of” indicates that the listed elements are required or mandatory, and that no other elements may be present.
“Mesenchymal stem cells (MSCs)” are multipotent stromal cells that can differentiate into a variety of cell types, including osteoblasts (bone cells), chondrocytes (cartilage cells), myocytes (muscle cells) and adipocytes (fat cells which give rise to marrow adipose tissue). MSCs are multipotent mesoderm-derived progenitor cells. The minimal criteria set by The International Society for Cellular Therapy is that MSCs are CD70, CD90, and CD105 positive, and CD34 negative. They can be found in nearly all tissues, preferably bone-marrow, peripheral blood, menstrual blood, salivary gland, skin and foreskin, synovial fluid, endometrium, dental tissue, adipose tissue and neonatal birth-associated tissues including placenta, umbilical cord, cord blood, amniotic fluid and amniotic membrane.
By “mesenchymal stem cells induced to differentiate into chondrocytes” refers to those mesenchymal stem cells wherein the differentiation to chondrocytes has been promoted by the method of the invention.
“Therapeutically effective dose or amount” of a composition comprising the MSCs of the invention is intended an amount that brings about a positive therapeutic response in a subject having tissue damage or loss, such as an amount that results in the generation of new tissue at a treatment site. The exact amount required will vary from subject to subject, depending on the species, age, and general condition of the subject, the severity of the condition being treated, mode of administration, and the like. An appropriate “effective” amount in any individual case may be determined by one of ordinary skill in the art using routine experimentation, based upon the information provided herein. For example, a therapeutically effective dose or amount is intended to be an amount that brings about a positive therapeutic response in a subject having cartilage damage or loss, such amount resulting in the generation of new cartilage at a treatment site (e.g., a damaged joint). For example, a therapeutically effective dose or amount could be used to treat cartilage damage or loss resulting from a traumatic injury or a degenerative disease, such as arthritis or other disease involving cartilage degeneration. Preferably, a therapeutically effective amount restores function and/or relieves pain and inflammation associated with cartilage damage or loss.
The terms “treatment” and “therapy”, as used in the present application, refer to a set of hygienic, pharmacological, surgical and/or physical means used with the intent to cure and/or alleviate a disease and/or symptom with the goal of remediating the health problem. The terms “treatment” and “therapy” include preventive and curative methods, since both are directed to the maintenance and/or reestablishment of the health of an individual or animal. Regardless of the origin of the symptoms, disease and disability, the administration of a suitable medicament to alleviate and/or cure a health problem should be interpreted as a form of treatment or therapy within the context of this application.
“Pharmaceutically acceptable carriers”: As used herein, “pharmaceutically acceptable carrier” or “pharmaceutically acceptable diluent” refers to an excipient that may optionally be included in the compositions of the invention and that causes no significant adverse toxicological effects to the patient. It comprises any and all solvents, dispersion media, coatings, antibacterial and antifungal agents, isotonic and absorption delaying agents, compatible with pharmaceutical administration. The use of such media and agents for pharmaceutically active substances is well known in the art. Acceptable carriers, excipients, or stabilizers are nontoxic to recipients at the dosages and concentrations employed and, without limiting the scope of the present invention, include: additional buffering agents; preservatives; co-solvents; antioxidants, including ascorbic acid and methionine; chelating agents such as EDTA; metal complexes (e.g., Zn-protein complexes); any type of scaffolds, biodegradable polymers, such as polyesters; salt-forming counterions, such as sodium, polyhydric sugar alcohols; amino acids, such as alanine, glycine, glutamine, asparagine, histidine, arginine, lysine, ornithine, leucine, 2-phenylalanine, glutamic acid, and threonine; organic sugars or sugar alcohols, such as lactitol, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, [alpha]-monothioglycerol, and sodium thio sulfate; low molecular weight proteins, such as human serum albumin, bovine serum albumin, gelatin, or other immunoglobulins; and hydrophilic polymers, such as polyvinylpyrrolidone.

### BRIEF DESCRIPTION OF THE FIGURES

FIG. 1. UC-MSCs pre-treated with oligomycin increase their suppressive potential compared to other OXPHOS inhibitors. Mouse Splenocytes were obtained from C57/BL6 mice and co-cultured with or without murine MSCs pre-treated with different OXPHOS inhibitors such as atovaquone, KCN, and oligomycin (6 or 24 hours pre-treatment) in the presence of the mitogen Concanavaline A (ConA). The analyses were performed using a flow cytometer and the proliferation was quantified in splenocytes after 3 days of activation. Proliferation of T-CD4+ (A) or CD8+ (B) cells with or without UC-MSCs pre-treated with the different OXPHOS inhibitors. N=3 Different mice performed in 3 independent experiments; ConA vs MSCs * p<0.05; ** p<0.01; *** p<0.001; Control MSCs vs Oligomycin pre-treated #p<0.05.

FIG. 2. Different inhibitors of the ATP synthase induce a similar enhancement in the immunosuppressive potential of MSCs. UC-MSCs were treated for 24 hours with multiple inhibitors of the ATP synthase complex. Then these pre-treated UC-MSCs were co-cultured with human peripheral blood-derived mononuclear cells (PBMC) for 4 days. Proliferation of CD4+ and CD8+ cells (A-B) was quantified by flow cytometry and graphed. N=3 different donors from 3 independent experiments * p<0.05.

FIG. 3. Different ATP-Synthase inhibitors enhance the chondroprotective properties of UC-MSCs. (A) Experimental design to test resistance to H₂O₂. (B) Representative density plot analysis of Annexin V and IP to identify live cells. The gated box represents the living chondrocytes after being coculture with or without H₂O₂ in the presence or absence of MSC pretreated or not with several ATP Synthase inhibitors. (C) Coculture of UC-MSCs treated with ATP synthase inhibitors, with chondrocytes in a semipermeable membrane of 0.4 μM diameter pores, significantly promotes the survival of chondrocytes in H₂O₂, compared to control UC-MSCs. N=3 different donors from 3 independent experiments—* p<0.05. OLN (oligomycin). Resv (resveratrol). Venta (venturicidin). PTSB (pterostilbene). TBT (Tributyltin). OLN+Resv (oligomycin and resveratrol).

FIG. 4. Oligomycin pre-treatment induces a glycolytic dependent metabolism, without affecting MSCs markers. (A) MSCs treated with ATP-Synthase inhibitors show a diminished oxidative phosphorylation, quantified as oxygen consumption rate (OCR), (B) and an enhanced glycolysis, shown as extracellular acidification rate (ECAR). (C) The overall metabolic switch towards a glycolytic dependent metabolism can be graphed as the ECAR/OCR ratio. (D) Lactate efflux is increased on MSCs treated with ATP-Synthase inhibitors as a secondary product of glycolysis. (E) Surface markers of UC-MSC were not affected after metabolic switch with oligomycin.

FIG. 5. UC-MSCs spontaneously differentiate into chondrocytes when pre-treated with oligomycin for 24Hrs. UC-MSCs were differentiated towards chondrogenic lineage. The generation of a three-dimensional tissue (micromass) was observed in UC-MSCs pre-treated with oligomycin and in those with differentiation media (A). The relative expression of the proteoglycan Aggrecan, which is typically expressed in hyaline cartilage, was quantified by qPCR. An increase in the relative expression of this gene was observed in UC-MSCs pre-treated with oligomycin compared to the untreated control (B), as well as in UC-MSCs that were induced with differentiation medium towards chondrocytes (C). When MSC on basal media (CTL) are compared to MSC treated with inhibitors of the ATP-synthase, these treated MSC present chondrogenic behaviour on the ratio of Collagen II vs Collagen I proportion. Moreover, some inhibitors such as resveratrol promote the MSC in basal media to behave as MSC that are in a differentiation media (D). There is a fold change in the production of collagen type 2 versus collagen type 1 when the MSC are treated with an inhibitor of the ATP-synthase (using basal media), such as oligomycin and resveratrol, when these inhibitors are compared to the normal ratio of MSC in basal media (CTL). Also, some inhibitors present the same behaviour than MSC in differentiation media (differentiation) (D).

FIG. 6. UC-MSCs pre-treated with oligomycin induce higher proliferation of chondrocytes. Experimental design to test proliferation rate of chondrocytes (A). UC-MSCs treated with ATP-synthase inhibitors significantly stimulate proliferation of chondrocytes, when co-cultured on a semipermeable membrane (B). N=4 different donors from 3 independent experiments—* p<0.05.

FIG. 7 UC-MSCs pre-treated with oligomycin facilitate the release of soluble chondroprotective factors. The production of hepatic growth factor (HGF) and fibroblast growth factor (FGF) was quantified on supernatants of UC-MSCs collected 24 hours post stimulation (control vs UC-MSCs treated with ATP-synthase inhibitors). UC-MSCs treated with ATP-synthase inhibitors show a small increase of both, HGF (A) and FGF (B). Similarly, when UC-MSCs pre-treated with oligomycin were co-cultured with chondrocytes in semipermeable membrane for 72h, we observed a slight increase of HGF (C) and FGF (D), compared to control UC-MSCs. N=3 different donors from 3 independent experiments; * p<0.05.

FIG. 8. Pre-treatment with oligomycin increases the therapeutic potential of MSCs in a model of delayed type hypersensitivity (DTH). A) Experimental design of the in vivo model of DTH. B) Graph of measurement of limb swelling (Paw Swelling) in mice with DTH treated or not with MSCs pre-incubated in the presence or absence of oligomycin. N=5 mice per experimental group performed at least 3 times independently—* p<0.05.

FIG. 9. Pre-treatment with oligomycin increases the survival rate of MSCs in a model of graft versus host disease (GVHD). A) Experimental design of the in vivo model of GVHD. B) Kaplan-Meier plot showing the survival rate of GVHD mice compared to UC-MSCs that were pre-treated or not with oligomycin. N=15 mice per experimental group performed at least 2 times independently—* p<0.05.

FIG. 10. MSCs treated with ATP-Synthase inhibitors exhibit an enhanced therapeutic effect on a murine osteoarthritis model. Experimental design of the in vivo model of murine osteoarthritis induced by Colagenase VII (A). Micro-Computed Tomography analysis of the knees of mice, revealed that mice that develop the disease present an increased bone mineral density (BMD), typical of the osteophyte formation associated to OA (B). UC-MSCs were effective preventing the development pf the disease (C).

FIG. 11. UC-MSCs pretreated with oligomycin promote their transmigration potential. GlycoStem cells were cultured on a semipermeable membrane with 8 μM diameter pores. Cells were seeded in serum-free medium in the upper chamber, while DMEM medium with 5% fetal bovine serum was added in the lower chamber to induce transmigration of the cells. Cell migration was quantified 8 hours (A and B) and 24 hours (C and D) later. UC-MSCs pretreated with oligomycin significantly increase their migratory capacity. N=3 different donors from 3 independent experiments * p<0.05.

FIG. 12. Pretreatment with Oligomycin enhances the immunosuppressive abilities of human MSCs independent on the origin. Human BM-MSCs, UC-MSCs or MenSC were coculture with freshly isolated PBMC activated with phytohemaglutinine in a 1 MSCs:20 PBMCs ratio. After 4 days proliferation was measured by quantifying the decrease in cell fluorescence on either T-CD4 (A) or T-CD8 (B) cells in PBMCs by FACS. Results are represented as mean and SD of 3 independent experiments using at least 3 different PBMC donors and 2 different BM-MSCs, MenSC and UC-MSCs donors. Statistical analyses used the non-parametric Friedman test comparing more than two groups (*, p<0.05; **, p<0.01; ***, p<0.005).

FIG. 13. ATP-Synthase inhibitor pretreatment exhibit an enhanced therapeutic effect on a murine osteoarthritis model. A) Experimental design of the in vivo model of murine osteoarthritis induced by Colagenase VII. B) Histological analysis of the knees of mice, revealed that mice that develop the disease present higher cartilage destruction as compared to non-OA mice (Sham). Moreover when mice were treated with 50.000 MSC under basal conditions ((OA+MSCbasal) no cartilage regeneration was observed. Meanwhile mice treated with 50.000 MSC that were pretreated with an ATP-Synthase, such as Oligomycin, (where the induction media was removed afterwards and the cells were washed with PBS) showed a significantly increased cartilage regeneration. C) Table that summarized the histological score mean value. A higher OA score value indicates a bigger cartilage degeneration. OA-mice treated with MSC that were incubated with oligomycin 24 h before injection showed a lower OA score representing a better cartilage regeneration. N=8 animals per experimental group. Data were analyzed using the kruskall wallis test for non-parametric values.

FIG. 14. ATP-Synthase inhibitor pretreatment of MSC displayed enhanced suppressive activity that is dose dependent. MSC were with different oligomycin concentration (0.1 μg/ml, 1 μg/ml and 10 μg/ml), the media was removed and the cells were washed and cultured with freshly isolated human mononuclear cells (PBMC) that were stained with cell trace violet. Proliferation of CD4+ and CD8+ was quantified after 4 days of co-culturing by flow cytometry and graphed. N=3 PBMC and * p<0.05.

FIG. 15. Oligomycin pretreatment of MSC display enhanced suppressive activity when preincubated for 6 and 24h. MSC were treated for 6 and 24 hours oligomycin. Then, the media was removed, the cells were washed and these pretreated MSCs were co-cultured during 3 days with freshly isolated murine CD4 that were stained with cell trace violet. Proliferation of CD4+ was quantified by flow cytometry and graphed. N=4 different mice and * p<0.05.

### DETAILED DESCRIPTION OF THE INVENTION

In a preferent embodiment, the invention refers to a method to obtain mesenchymal stem cells (MSC) with enhanced activity for the treatment of osteoarticular diseases or trauma, wherein the method comprises providing MSC; culturing MSC in a induction media supplemented with 0.01 to 100 μg/ml of an ATP-synthase inhibitor; and removing the induction media to obtain MSC with enhanced activity.

Where the ATP-synthase inhibitor is selected from the list comprising: Oligomycin, Tentoxin, Efrapeptins, Substrate analogs (DCCD, CMCD, EEDQ,NBD-Cl (βE), Azide), Aurovertins, Asteltoxin, Resveratrol, Piceatannol, α-Helical basic peptides, Bz-423, Estrogens, Angiostatin, Enterostatin, Citreoviridin, Quinacrine mustard, Bathophenanthroline-metal chelates, Venturicidin, DCCD, NCCD, R207910, Organotin compounds, pterostilbene, tributyltin or Ossamycin, or combinations thereof. Specially, the ATP-synthase inhibitor is selected from the list comprising: Oligomycin, Venturicidin, Resveratrol, Piceatannol, Tributyltin and Pterostilbene, or combinations thereof.

The mesenchymal stem cells used in the method according to the invention are derived from: umbilical cord, adipose tissue, menstrual fluid, bone marrow, dental pulp, blood, endometrial tissue, peripheral blood, placental tissue or they are induced pluripotent stem cells.

Therefore, the method of the invention comprises providing MSC; culturing MSC in a induction media supplemented with 0.01 to 100 μg/ml of an ATP-synthase inhibitor, incubating for 2 to 48 hours; removing the induction media, to obtain MSC with enhanced activity. In a preferred embodiment the MSC are incubated for 24 hours. In another preferred embodiment, the induction media is supplemented with 0.1 to 10 μg/ml of an ATP-synthase inhibitor.

The invention also refers to mesenchymal stem cells with enhanced activity for the treatment of osteoarticular diseases or trauma wherein the MSC are obtained by the method of invention i.e. treated with an induction media supplemented with 0.01 to 100 μg/ml of an ATP-synthase inhibitor, and then removing the induction media. Where these MSC have enhanced immunosuppression, chondroprotection and chondrodifferentiation properties, cell migration and cell transmigration ability and enhanced capability to promote the proliferation of chondrocytes.

The invention also refers to a pharmaceutical composition comprising the mesenchymal stem cells of the invention and pharmaceutically acceptable carriers. Where the mesenchymal stem cells have enhanced immunosuppression, chondroprotection and chondrodifferentiation properties, cell migration and cell transmigration ability and enhanced capability to promote the proliferation of chondrocytes.

Moreover, the invention refers to the use of these pharmaceutical compositions in the treatment of osteoarticular diseases or osteoarticular trauma. Specially in the regeneration of damaged cartilage or in the generation of new cartilage. Finally the invention refers to a method for the regeneration of damaged cartilage or for the generation of new cartilage, which comprises the administration of a therapeutically effective amount of the MSCs according to the invention.

### Example 1. Method of Invention

Umbilical cord derived Mesenchymal stem cells (UC-MSCs) was cultured in DMEM medium supplemented with 10% FBS, Pen/Strept and glutamine (DMEM-10) until reaching 80% confluence when the culture medium is removed and washed once with buffered saline solution (PBS). Then the “glycolytic stimulation” was prepared by adding to the UC-MSCs fresh DMEM-10 medium supplemented with an ATP-Synthase inhibitor, for example, an oligomycin solution that contains the A, B, C isomers at a final concentration of 1 μg/ml. The medium containing the ATP-Synthase inhibitor was removed and the cells are washed with buffered saline solution (PBS). Then, we can obtained the MSCs of the invention (UC-MSCs treated with ATP-synthase inhibitors) after 6-24 hours of incubation, reaching the maximum effect after 24 hours of incubation.

MSCs treated with ATP-synthase inhibitors according to the invention consist in a cell product that presents a glycolytic metabolic state that promotes its therapeutic potential for the treatment of pathologies such as osteoarthritis, rheumatoid arthritis among other autoimmune disease. In order to determine the efficiency of MSCs treated with ATP-synthase inhibitors, the inventors first evaluated the glycolytic status of UC-MSCs using biochemical parameters such as lactate production and glucose consumption and also if possible, the metabolic status of MSCs treated with ATP-synthase inhibitors was evaluated in real time by measuring the oxygen consumption rate (OCR) or the extra cellular acidification rate (ECAR) by the Seahorse™ technology. Moreover, different functional assays were carried out in order to compare the efficacy of MSCs treated with ATP-synthase inhibitors as compared to non-treated UC-MSCs including evaluation of the differentiation potential towards chondrocytes, viability assays of MSCs, chondroprotection assays, immunosuppression assays and in vivo evaluation using joint inflammation murine models.

### Example 2. Results. In Vitro and In Vivo Assays

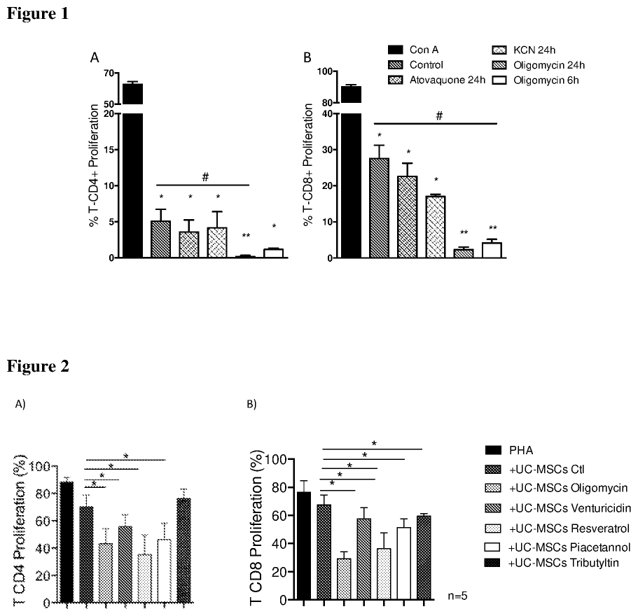

The present invention shows that the specific inhibition of the ATP-Synthase effectively enhanced the immunosuppressive potential of UC-MSCs (FIG. 1). This was further studied comparing multiple families of inhibitors of the ATP-Synthase complex. It was observed that all inhibitors tested were able to enhance the immunosuppressive function of UC-MSCs (FIG. 2), validating the hypothesis of the inventors that any inhibitor of the ATP-Synthase will enhance the potential of UC-MSCs. Moreover, pre-treated UC-MSCs display a variety of enhanced properties, which suggests a promising therapeutic approach for the treatment of autoimmune diseases.

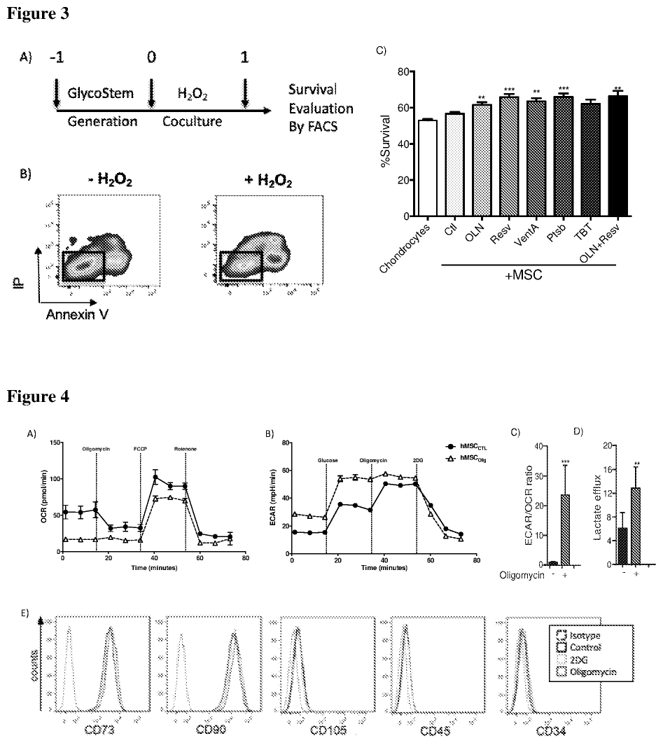

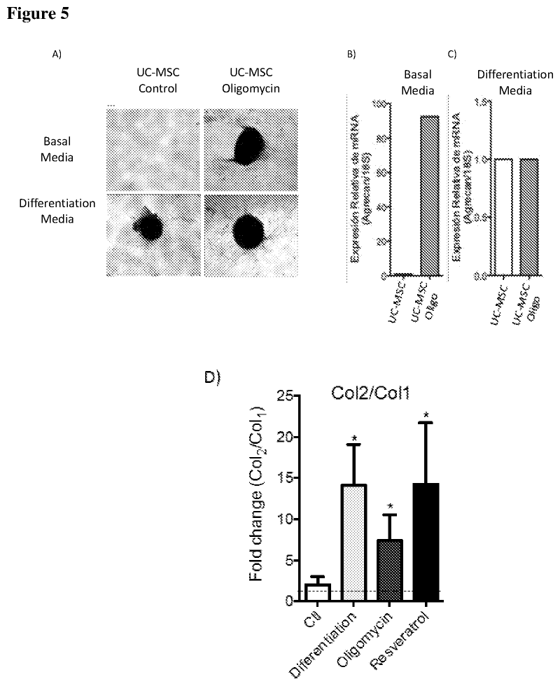

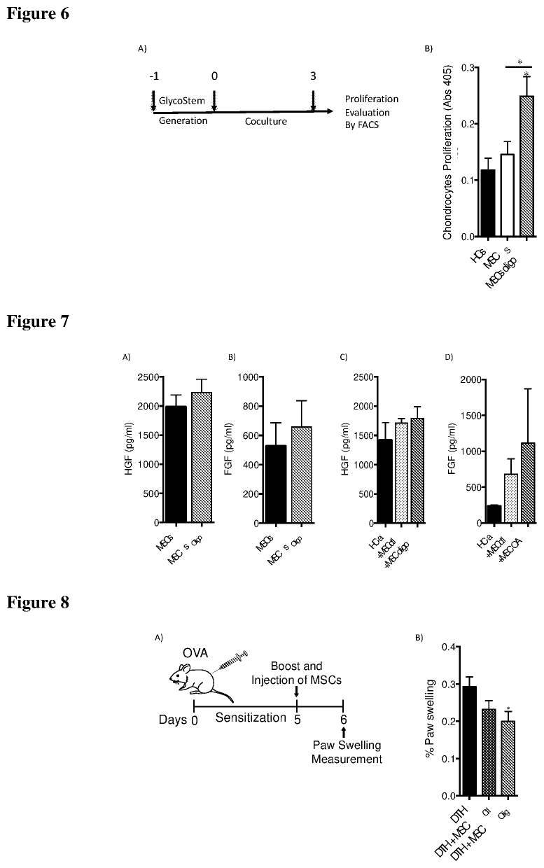

UC-MSCs treated with the different ATP-Synthase inhibitors show an enhanced chondroprotective effect, improving the viability of chondrocytes treated with hydrogen peroxide (FIG. 3), validating the hypothesis of the inventors that any inhibitor of the ATP-Synthase will enhance the chondroprotective effect of MSCs. Specifically, oligomycin seems to exhibit the most potent effects on MSCs, inducing a strongly glycolytic-dependent metabolism, without changing the “mesenchymal” profile of UC-MSCs (FIG. 4). Crucial for the treatment of any osteoarticular disease is the regeneration of functional cartilage. Remarkably, oligomycin pre-treatment stimulates the spontaneous differentiation towards a functional three-dimensional cartilage, which expresses high levels of Collagen II, main component of hyaline cartilage (FIG. 5), validating the hypothesis of the inventors that an inhibitor of the ATP-Synthase such as oligomycin will enhance the chondrodifferentiation potential of MSCs. Similarly, in order to induce regeneration of functional cartilage, proliferation of local chondrocytes is a key target to facilitate cartilage regeneration. The addition of oligomycin-pre-treated UC-MSCs shows a significant improvement in proliferation of chondrocytes (FIG. 6), validating the hypothesis of the inventors that an inhibitor of the ATP-Synthase such as oligomycin will enhance the proliferation of chondrocytes. This increased chondroprotection property is related to the secretion of growth factors, like HGF and FGF (FIG. 7).

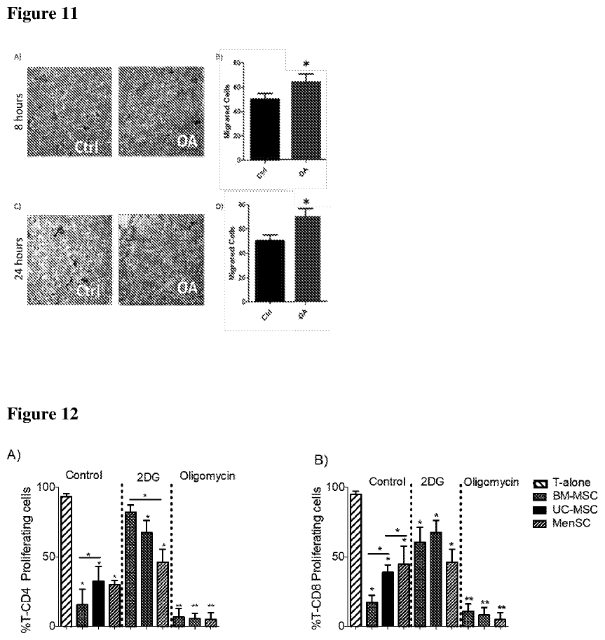

Additionally, the inventors proved that the transmigration potential, typically used for migration test, increased in MSCs when they were treated with an ATP-synthase inhibitor (FIG. 11).

It is worth mentioning, that the inventors also demonstrated that a pre-treatment with an ATP-synthase inhibitor enhances the immunosuppressive abilities of human MSCs independent on the origin, human BM-MSCs, UC-MSCs or MenSC enhanced their immunosuppression abilities (FIG. 12).

Moreover, the successful in vitro results obtained as explained above, were confirmed in vivo, using a murine model.

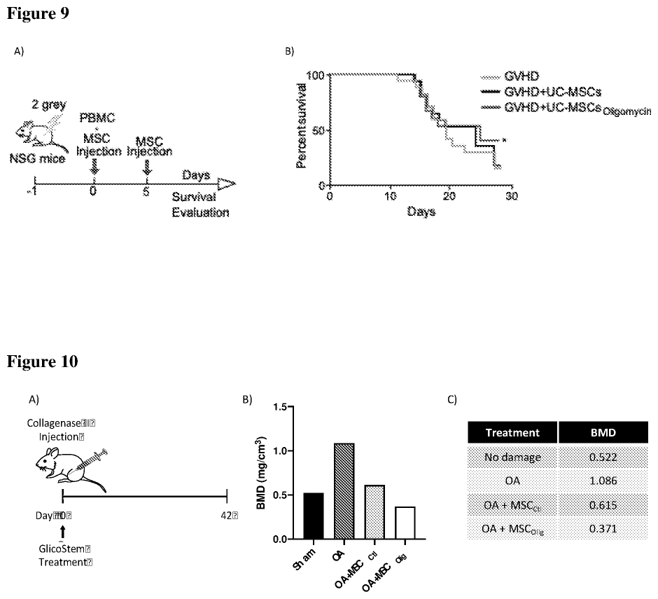

The MSCs treated with ATP-synthase inhibitors were able to diminish inflammation of the joints of mice induced with DTH (FIG. 9), to improve the survival in the murine model of GVHD (FIG. 8), and to significantly induce the regeneration on an osteoarthritis murine model (FIG. 9). Therefore, the inventors have proved the therapeutic potential of MSCs pre-treated with ATP-Synthase inhibitors for their use in autoimmune and/or osteoarticular diseases.

### Example 3. Results. In Vivo Osteoarthritis Model

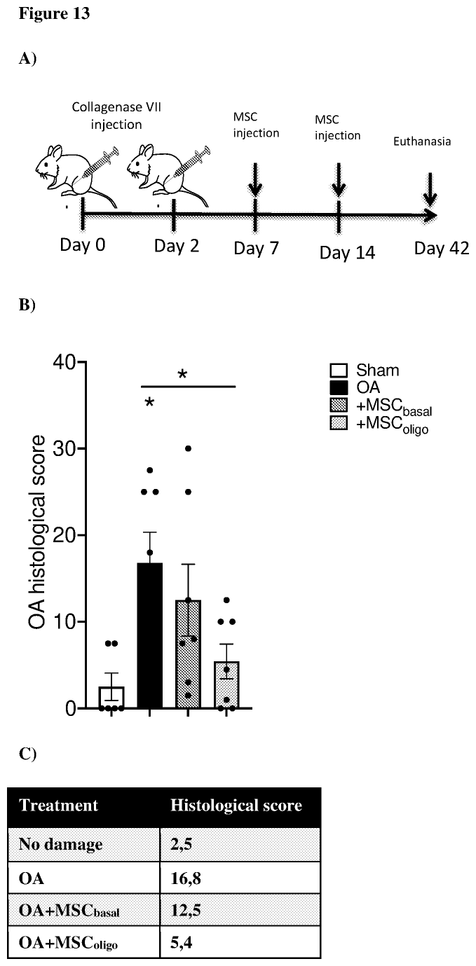

The present Invention shows that the specific inhibition of the ATP-Synthase effectively enhanced the therapeutic effect on a murine osteoarthritis model (FIG. 13). The inventors developed an in vivo assay using a murine osteoarthritis model, in this experiment the osteoarthritis was induced by Colagenase VII at day 0 and 2, then at day 7 and 14 an injection of MSCs and MSCs pretreated with an ATP-Synthase inhibitor (where afterwards, the media with ATP-synthase inhibitors was removed and the MSCs were washed) was applied as shown on FIG. 13. A. At day 42 the inventors finished the experiment and took knee samples for histological analysis as shown in FIGS. 13.B and 13.C.

The histological analysis of the knees of mice, revealed that mice that develop the disease presented higher cartilage destruction as compared to non-OA mice (Sham), proving that the inventors have developed a successful murine model. Moreover, when mice were treated with 50.000 MSC under basal conditions (OA+MSC_{basal}) no cartilage regeneration was observed, while mice treated with 50.000 MSC that were pretreated with an ATP-Synthase, such as Oligomycin, showed a significantly increased cartilage regeneration.

Therefore, according to the results presented on FIGS. 13.B and 13.C the inventors have proven that the use of an ATP-Synthase inhibitor enhance the therapeutic effect of MSC for the treatment of an osteoarticular disease.

### Example 4. ATP-Synthase Inhibitor Pretreatment of MSC Display Enhanced Suppressive Activity that is Dose Dependent

The present Invention shows that the ATP-Synthase inhibitor pretreatment of MSC displayed enhanced suppressive activity that is dose dependent. The inventors developed and immunosuppression experiment where the MSCs were pretreated with different concentrations of ATP-Synthase inhibitor, such as Oligomycin, more specifically at 0.1 μg/ml, 1 μg/ml and 10 μg/ml. After the pretreatment, the media with ATP-synthase inhibitor was removed, the MSCs were washed, and the MSC were cultured with freshly isolated human mononuclear cells (PBMC) that were stained with cell trace violet.

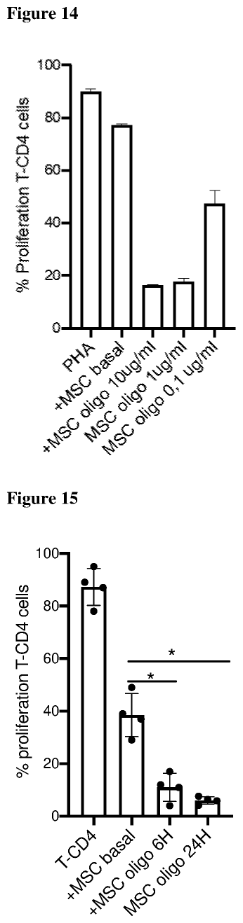

As shown on FIG. 14, the higher suppression was observed with the concentration of 1 μg/ml of ATP-Synthase inhibitor. Therefore, the inventors have proved a range of concentration for the use of an ATP-Synthase inhibitor from 0.1 μg/ml to 10 μg/ml, observing the highest effect at 1 μg/ml of ATP-Synthase inhibitor.

### Example 5. ATP-Synthase Pretreatment of MSC Display Enhanced Suppressive Activity when Preincubated for 6 and 24h

The present invention shows that the ATP-Synthase inhibitor pretreatment of MSC display enhanced suppressive activity when is pretreated for 6 and 24 hrs. The inventor developed an incubation experiment were the MSC were treated for 6 and 24 hours with an ATP-Synthase inhibitor, such as Oligomycin. After this treatment, the media with ATP-synthase inhibitor was removed, the MSCs were washed, and the MSCs were co-cultured during 3 days with murine freshly isolated CD4 and their effects were compared. As shown in FIG. 15, the ATP-Synthase inhibitor enhanced the suppressive activity of MSCs with a 6 or 24 hours pretreatment, moreover the 24 hours pretreatment exhibits the highest enhancement of the MSCs suppression activity.

Therefore, the inventors have proven that treatment with the ATP-Synthase inhibitor can be developed during a 6 to 24 hours in order to enhance the activity of MSCs.

1-17. (canceled)

18. Method to obtain mesenchymal stem cells (MSC) with enhanced activity for the treatment of osteoarticular diseases or trauma, wherein the method comprises providing MSC; culturing MSC in a induction media supplemented with 0.01 to 100 μg/ml of an ATP-synthase inhibitor; and removing the induction media to obtain MSC with enhanced activity; wherein the ATP-synthase inhibitor is selected from: Oligomycin, Tentoxin, Efrapeptins, Substrate analogs (DCCD, CMCD, EEDQ,NBD-Cl (βE), Azide), Aurovertins, Asteltoxin, Piceatannol, α-Helical basic peptides, Bz-423, Estrogens, Angiostatin, Enterostatin, Citreoviridin, Quinacrine mustard, Bathophenanthroline-metal chelates, Venturicidin, DCCD, NCCD, R207910, Organotin compounds, tributyltin or Ossamycin, or combinations thereof.

19. Method according to claim 18, wherein the ATP-synthase inhibitor is selected from the list comprising: Oligomycin, Venturicidin, Piceatannol, Tributyltin and or combinations thereof.

20. Method according to claim 18, wherein the mesenchymal stem cells are derived from: umbilical cord, adipose tissue, menstrual fluid, bone marrow, dental pulp, blood, endometrial tissue, peripheral blood, placental tissue or they are induced pluripotent stem cells.

21. Method according to claim 20, wherein the method comprises providing MSC; culturing MSC in a induction media supplemented with 0.01 to 100 μg/ml of the ATP-synthase inhibitor; incubating for 2 to 48 hours; and removing the induction media to obtain MSC with enhanced activity.

22. Method according to claim 21, wherein the MSC are incubated for 24 hours with the ATP-synthase inhibitor.

23. Method according to claim 21, wherein the induction media is supplemented with 0.1 to 10 μg/ml of the ATP-synthase inhibitor.

24. Mesenchymal stem cells with enhanced activity for the treatment of osteoarticular diseases or trauma wherein the MSC are obtained by the method of claim 18, treating them with an induction media supplemented with 0.01 to 100 μg/ml of the ATP-synthase inhibitor, and then removing the induction media.

25. Mesenchymal stem cells of claim 24 wherein the MSC have enhanced immunosuppression, chondroprotection and chondrodifferentiation properties, cell migration and cell transmigration ability and enhanced capability to promote the proliferation of chondrocytes.

26. Mesenchymal stem cells of claim 24 wherein the ATP-synthase inhibitor is selected from the list comprising: Oligomycin, Venturicidin, Piceatannol and Tributyltin.

27. Mesenchymal stem cells of claim 24 wherein the mesenchymal stem cells are derived from: umbilical cord, adipose tissue, menstrual fluid, bone marrow, dental pulp, blood, endometrial tissue, peripheral blood, placental tissue or they are induced pluripotent stem cells.

28. Pharmaceutical composition comprising the mesenchymal stem cells according to claim 24 and pharmaceutically acceptable carriers.

29. Pharmaceutical composition of claim 28 wherein the mesenchymal stem cells have enhanced immunosuppression, chondroprotection and chondrodifferentiation properties, cell migration and cell transmigration ability and enhanced capability to promote the proliferation of chondrocytes.

30. Use of Pharmaceutical composition of claim 28 in the treatment of osteoarticular diseases or osteoarticular trauma.

31. Use of Pharmaceutical composition of claim 28 in the regeneration of damaged cartilage or in the generation of new cartilage.

32. Method for the regeneration of damaged cartilage or for the generation of new cartilage, which comprises the administration of a therapeutically effective amount of the MSCs according to claim 24.
